(19) United States
(12) Patent Application Publication
(10) Pub. No.: US 2024/0051741 A1
(43) Pub. Date: Feb. 15, 2024

(54) INDUCTIVELY HEATABLE WASTE RECEPTACLE AND DOCKING STATION FOR INDUCTIVELY HEATABLE WASTE RECEPTACLE FOR INDUCTIVE STEAM DECONTAMINATION

(71) Applicant: Tomas PINK, Beromünster (CH)

(72) Inventor: Tomas PINK, Beromünster (CH)

(21) Appl. No.: 18/259,600

(22) PCT Filed: Dec. 22, 2021
(86) PCT No.: PCT/EP2021/087263

§ 371 Date: Jun. 28, 2023

(30) Foreign Application Priority Data
Dec. 28, 2020 (EP) 20217459.5
Aug. 10, 2021 (EP) 21190683.9
Aug. 10, 2021 (EP) 21190687.0

(51) Int. Cl. B65F 7/00; B65F 1/14

(52) U.S. Cl. CPC B65F 7/00; B65F 1/141; B65F 2210/168; B65F 2210/181

(57) ABSTRACT

A device, use, and method for steam sterilisation and/or steam decontamination and/or sterilisation of the contents of a container. The device, method, and use enable efficient, simple, and reliable sterilisation and/or decontamination. The device makes it possible to collect and sterilise a wide variety of solid, semi-solid, or liquid items and to decontaminate various types of biologically contaminated objects effectively and reproducibly. The system includes in particular of two parts. One of them is the docking station; a unit which can be used again and again for many sterilisation/decontamination cycles. The container itself, which is brought into the docking station for sterilisation/decontamination, is used for collecting the items and is disposed of, in particular incinerated, after sterilisation/decontamination. However, the container has functional elements which interact with the docking station to ensure sterilisation/decontamination on, in particular at least one inductively heatable heating element.

The present invention relates to a device, a utilization and a method for steam decontamination and/or steam sterilization of the contents of a container.

Waste bins with UV lamps are known from CN 108100532A.

It is also known, inter alia, from US20180055 595A1, WO201 6109416A1 and WO201 7129848A1, to position waste containers with an opening in an autoclave and to sterilize their contents there by steam penetrating from the outside.

Also known from CN204433530U and CN104590793A is a trash can which has an electric heater, a pressure gauge, a water injection port, a water level meter and a power supply port.

In addition, an autoclave with connections for further autoclaving chambers is known from EP 3 495 001 A1.

It is also known from US 201 1 21 1.989 A1 to inductively heat metallic material in a container or, as described in US 2003 23.05 67 A1, to inductively heat liquids in a metal pipe in order to vaporize them and discharge the vapor into a container.

Existing technologies for efficient steam sterilization and/or steam decontamination are expensive, but also relatively bulky and heavy, fragile and not easy to transport. During transport, they can be damaged or, for example, the calibration parameters of the sensors can be affected, which can have a major impact on the efficiency of the process. In addition to the electrical connection, other energies and media are also required, e.g. deionized water, tap water, compressed air, external steam supply. The systems also require regular maintenance and the replacement of various components after a certain number of sterilization/decontamination cycles.

Other solutions like those from the CN108100532A, CN204433530U, CN 104590793A also offer insufficient sterilization effectiveness.

The devices available on the market can decontaminate hazardous waste, but they do not themselves meet the hygiene requirements (EHEDG) and are not suitable for damp environments or outdoor use, for example. Their construction has no IP classification and if they are contaminated from the outside it is not really possible to decontaminate them.

Other, simple methods, e.g. chemical decontamination/disinfection, cannot offer the high efficiency and change the chemical composition of the waste. Which can lead to further problems.

The object of the invention is to specify a device, a method and a utilization for efficient, simple and reliable sterilization/decontamination.

The invention offers a technical solution that makes it possible to safely and reproducibly sterilize/decontaminate various types of objects, solids, semi-solids and liquids with high efficiency, including objects, that during the process, due to the sterilization and/or or decontamination temperature, go through their phase transition (melt).

In particular, the system consists of two parts. One of these is the docking station, a unit that can be used again and again for many sterilization/decontamination cycles. This unit is connected to the power supply. It preferably communicates with the user (HMI), contains control electronics and other elements that are required for the operation of the disposable part, the container.

The container, in particular waste container, itself, which is advantageously brought into the docking station for the sterilization/decontamination, is used to collect the items, in particular waste collection, and disposed of after sterilization/decontamination, in particular incinerated. The container has functional elements that interact with the docking station to ensure sterilization/decontamination.

However, steam is not transferred into the container from the outside. Rather, the container in particular remains closed and contamination of the environment is thus effectively prevented.

The object is achieved by a system using, among other things, a container, in particular a waste container, having a first cavity enclosed on all sides, in particular by a wall, floor, intermediate floor and/or lid, among other things. The first cavity is in particular surrounded by a wall, in particular enclosing on four sides, and/or at least one cover, in particular closing on a fifth side, and/or at least one enclosing, in particular on a sixth side opposite the fifth, a floor and/or intermediate floor. The container advantageously has at least one, in particular precisely one, second cavity which is adjacent to the first cavity or located in it, in particular under an intermediate floor, within which at least one first heating element is arranged. In particular if there is no second cavity, the first heating element can also be arranged in the first cavity.

In particular, the intermediate floor is ring-shaped guided. In particular, the at least one second cavity is arranged in a lower section of the first cavity, in particular in a ring-shaped manner around a lower section. In particular, the container is set up to seal the first and/or second cavity and/or their entirety in a fluid, water, gas and/or airtight manner with respect to the environment. The container is characterized in that the at least one first heating element is an inductively heatable heating element. The at least one second cavity is preferably sealed in a watertight, fluid, gas and/or airtight manner and/or means are provided for transferring vapor from the at least one second cavity into the at least one first cavity. The means for transfer can be implemented, for example, in that at least one, in particular at least four, in particular with and/or by overpressure and/or with and/or by the temperature/heat in at least one second cavity opposite the first cavity, passage(s) opening(s), in particular in an intermediate floor arranged between the first and at least the second cavity. However, the intermediate floor can also be realized by a porous plate, a lattice, net, struts or by a combination of such a means with a foil. In the case of a lattice or net, the passages (meshes) in the lattice represent the means of transfer.

For example, a non-vapor, water- and/or air-tight separation is preferred, but which protects the second cavity from the effects of force from the contents, in particular waste, in the first cavity. This can be done, for example, by means of a grid, struts or a stretched net in combination with a watertight vessel which has a film wall as a wall, in particular for separation. The vessel can be formed, for example, by a closed film bag containing water in particular. The vessel is arranged in particular on the side of the partition facing away from the first cavity. The vessel is designed in particular in such a way that it opens due to the effect of temperature/heat and/or the resulting increase in pressure, for example by melting or bursting, particularly at a temperature below 80° C., particularly at normal external pressure.

For example, two or one structural element(s) can serve as an intermediate floor, for example a grid and a foil. In principle, the intermediate floor or at least one of its structural elements should not, or at least not significantly, deform under the influence of heat up to at least 140° C. and should not melt and/or not absorb any water or water vapor and/or be inert to water vapor and water, in particular not hygroscopic.

Plastic netting, plastic grids, aluminum plates with holes, ceramic plates, porous graphite, graphite felt can be used as material for the intermediate floor.

Such an arrangement, in which the function of holding the water and protecting the collected items, in particular the rubbish, from mechanical effects on the holding of the water are realized in separate means, can be produced in a particularly simple and material-saving manner.

The opening takes place in particular by overpressure, in particular of at least 0.5 barg and/or a maximum of 3 barg and/or by temperature/heat, in particular at a temperature in the range between 70° C., in particular between 80° C., in particular between 95° C., and 140° C. This can ne done for example by bursting and/or melting a seal and/or closure, for example the film. But it can also be done by other precautions such as an electrically controllable valve. In particular, the heating of the at least one second cavity begins when the means for transferring are closed and/or when the at least one second cavity is completely closed. In particular, the means for transferring and/or the at least one second cavity remain completely closed until a temperature of at least 40° C. and/or an overpressure is reached in the at least one second cavity of at least 0.2 barg prevails and/or at least during transport and/or during transport before filling in items/contents, in particular waste. The pressure is always determined in relation to the first cavity.

However, the second cavity can also be open.

In an alternative embodiment, the water can be enclosed in a third hollow space, in particular of the container, and this can be opened before or by the heating and/or be designed to be opened by the heating and/or the overpressure caused thereby. In particular, the container is designed in such a way that the water from the third cavity flows onto and/or around the first inductively heatable heating element when the third cavity is opened. In particular, the third cavity can be formed by a bag or capsule that is located in the container and/or waste container.

In a simple embodiment, however, water can also simply be filled into the first cavity in which the first inductively heatable heating element is located, in particular before the content, in particular waste, is filled in.

The task is also solved by a docking station and a combination of docking station and container, in particular waste container. The docking station is set up to receive a container, in particular as described above, the docking station being set up to generate an electromagnetic field for heating an inductively heatable heating element and means for, in particular supporting, enclosing a container, in particular to prevent it from bursting at overpressure in the container up to at least 3 barg and/or as a safety barrier and/or insulation.

In particular, the object is achieved by a system having at least one docking station according to the invention and at least one, in particular a large number of, inventive containers, in particular as described above.

An inductively heatable heating element is understood to mean, in particular, a resistively heating element which is set up to induce a current in it, in particular a ring current, by means of an electromagnetic field, which then leads to resistive heating. The heating element is therefore set up in particular to convert electrical current into heat. A current, in particular a ring current, which is used to generate heat is preferably induced in the heating element by means of an electromagnetic field.

However, the object is also achieved by using and/or utilizing at least one first inductively heatable heating element in a cavity, in particular a second or first cavity, with water to generate an overpressure in the enclosed cavity and in particular for opening at least one, in particular at least four, passage(s), in particular by means of temperature/heat and/or excess pressure, and introducing and/or generating water vapor in a, in particular adjacent, receiving space, in particular for waste, in particular first hollow space for steam sterilization of the receiving space and/or the contents in the waste receiving space and/or receiving space. The introduction takes place in particular through the at least one passage.

The object is also achieved by a method for decontamination and/or sterilization, in particular as part of disposal and/or for waste disposal, sterilization and/or decontamination, with at least a first inductively heatable heating element in a cavity, in particular the first or second cavity to heat with water by means of a field generated by means of at least one induction coil for evaporating the water, so that an overpressure is generated in the enclosed, in particular first and/or second, cavity and, in particular, by opening at least one, in particular a number of passages, particularly through the temperature/heat and/or the overpressure in the enclosed cavity, and thereby water vapor in an adjoining reception space, in particular waste reception space, in particular first cavity, for steam sterilization of the reception space and/or the content in the reception space, in particular waste, is transferred and/or generated and in particular the receiving space and the enclosed cavity are then burned, the induction coil being used to heat another inductively heatable heating element, in particular another container according to the invention.

Water is advantageously heated in the second cavity by means of the first inductively heatable heating element and an overpressure is generated and steam is transferred to the receiving space, in particular waste receiving space.

In a simple configuration, the water can also be heated and evaporated in the receiving room itself by a first inductively heatable heating element arranged there and an overpressure can be generated directly in the receiving room.

Preference is therefore given to at least a first inductively heatable heating element in a cavity, in particular a second cavity, with water to generate excess pressure in the enclosed cavity and in particular to open at least one passage(s) and introduce water vapor into an adjacent receiving space, in particular the first cavity, used for steam sterilizing the receiving space and/or the contents located in the receiving space. The first cavity can, in particular in a simple embodiment, also represent the receiving space.

In particular, the use and/or the method is carried out with a container according to the invention, in particular a waste bin, a system and/or a docking station according to the invention. In particular, is/are the container, the docking station and/or the system set up for performing the method.

The enclosing wall can be arranged, for example, as a cylinder, with a round or square or polygonal cross-section. It can be designed in one or more parts. The cover, floor and/or intermediate floor can, for example, be firmly connected to the wall and/or be made in one piece with it. Advantageously, the wall and intermediate floor and/or the lower base are made in one piece. This increases stability. In particular, the wall, the intermediate floor, the floor and/or the cover are double-walled. This increases the thermal insulation.

Waste is in particular biologically contaminated waste, in particular e.g. according to Art 3 RL 2008/98/EG. An electrically conductive ring, an electrically conductive plate or other suitable electrically conductive structure, for example, can be used as the inductively heatable heating element.

By hermetically sealed it is to be understood in particular that the container is airtight and/or gas-tight with mechanical support from the outside at least up to an internal pressure of 1 barg, in particular at 130° C., in particular up to an internal pressure of 3 barg, in particular at 130° C.

The passages are designed in particular as non-return valves which close when there is an overpressure in the first cavity compared to the at least one second cavity. In another preferred, particularly simple embodiment, they are formed from a seal that melts and/or bursts during the generation of steam in the at least one second cavity, in particular in the temperature range from 100 to 120° C.

The passages are designed in particular as nozzles, which are in particular directed to generate a directed jet of steam in the first cavity.

This allows good mixing to be achieved in the first cavity during sterilization.

The docking station can advantageously be designed both as a “standalone unit” or as a “built-in unit” for installation in cabinets, tables and/or containers and/or built into a cabinet, table and/or container.

In this document, “single-use” material is understood to mean a material or a mixture of materials which, when disposed of, in particular landfilled and/or incinerated, in particular when incinerated at temperatures in the range of 800-1 150° C., in particular 800° C. and 900° C., and/or in the case of fluidized bed combustion and/or grate firing, do not release any hazardous by-products or emissions and either burn without residues and/or decompose or leave only residues which are harmless to humans and the environment and/or those which meet the requirements of the BlmSchG, of the BlmSchV and/or Industrial Emissions Directive 201017 5/EU (Integrated Pollution Prevention and Control) in the latest and/or valid version at the time of application, in particular for combustion at temperatures in the range of 800-1 150° C., in particular 800° C. and 900° C., and/or in the case of fluidized bed combustion and/or grate firing.

Preferably, they do not contain rare elements or other valuable materials, in particular rare or valuable materials whose value is reduced by more than 50% by the effect of heat, in particular permanent magnets. Preferred materials are plastics, enamels, ceramics, steel, tungsten carbide, titanium nitride, aluminum oxide, metal and semi-metal nitrides, metal carbides, metal and semi-metal oxides, glass and/or carbon composite, glass and/or carbon fibers, aramide, aramide resin and/or aluminum and aluminum alloys, magnesium alloys, preferably plastics. Suitable plastics include polypropylene, polyethylene, polyvinyl acetate, polyethylvinyl acetate, PEEK, PET, polyamides and polyimides.

The container particularly advantageously contains at least one sensor, in particular at least one temperature and/or pressure sensor, with at least one sensor being set up for the wireless transmission of measured values and/or for the wireless power supply, in particular for the measurement and/or transmission of measured values and/or the at least one sensor and/or its means for transmitting measured values and/or for wireless power supply are arranged in the upper half of the container. With such sensors, the process can be monitored and/or controlled and/or regulated based thereon. In particular, the transmission means for transmitting the measured values, for example at least one antenna, are located at least 10 cm away from the inductively heatable heating elements, in particular in the upper half, in particular in the upper fifth, of the container. At the top is in particular the end of the container which is remote from the at least one first inductively heatable heating element and/or the opposite end. In this way, interference from the induction can be avoided. The at least one sensor is connected to the means for transmission, in particular by means of at least one cable and/or electrical conductor. At least one pressure and/or temperature sensor for measuring the pressure and/or temperature is particularly advantageous arranged in the top third, in particular in the top fifth, of the first cavity.

At least one temperature sensor for measuring the temperature in the bottom third, in particular in the bottom fifth, of the first cavity is preferably provided. The process can thus be monitored and/or controlled and/or regulated particularly reliably. A liquid level sensor for determining the liquid level in the first cavity in the container and/or docking station is arranged with particular advantage.

The at least one second cavity is advantageously at least partially filled with water. It is filled with water, in particular before the container is filled with items/contents, in particular waste. In particular, the water is completely enclosed in the at least one second cavity. In particular, the at least one second cavity has no openings to the outside. In particular, apart from the means for transferring, it has no opening means. In particular, at least 0.80 ml, in particular at least 2 ml, of water are present in the at least one second cavity per 1 liter of volume of the first cavity. In particular, a maximum of 5 ml, in particular a maximum of 100 ml, of water is present in the at least one second cavity per 1 liter of volume of the first cavity. If there are several second cavities, the amounts of water in all the second cavities must be added up for this consideration. Sterilization/decontamination can be achieved particularly efficiently with these amounts of water.

The container and/or the wall, the floor, the intermediate floor and/or the lid are made of “single-use” material, in particular plastic, in particular polypropylene and/or “glass fiber filled” polypropylene, with particular advantage. This is particularly resource-friendly and easy to manufacture.

The container is preferably designed in such a way that it cannot withstand an internal pressure of 3 barg at 130° C. without external support. This saves on construction material of the container, which in turn is particularly resource-saving and the container is therefore easy to handle and transport. The resistance to 3 barg and more can be achieved with support, especially with the docking station.

The waste container is preferably designed in such a way that, with mechanical support and/or enclosure, in particular by the enclosure of the docking station, it resists an internal pressure of at least 3 barg at 130° C. in the first and/or second cavity. This enables reliable steam sterilization/decontamination.

The container particularly advantageously has a pressure transmission area, in particular a membrane, in particular in the wall, which is designed in such a way that it makes it possible to measure the internal pressure in the first and/or second cavity from the outside. For this purpose, the docking station has in particular a pressure sensor to be arranged against this pressure transmission area during the process in order to measure the pressure in the first cavity. In particular, this pressure transmission area is designed for the pressure sensor to be pressed from the outside against it. This means that the pressure sensor can be used multiple times with different containers.

The container preferably has at least one second inductively heatable heating element. In particular, it is designed in such a way that condensate flowing down the walls of the container inside the container is collected and guided to the at least one first or second heating element so that it can be heated with the at least one first or second heating element, in particular with this is brought into contact. This allows the evaporation of condensate and/or liquid in the first cavity to be reliably achieved and the effectiveness of the steam sterilization/decontamination to be increased.

The first and/or second heating element is/are preferably made of “single-use” material, of metal and/or metal alloy, in particular it/they are made of aluminum or aluminum alloy and/or the container is made exclusively of Single-use” material.

The container, in particular the waste container, particularly advantageously has a closable slot, in particular in the lid and/or in the wall. This makes filling easy, but contamination of the environment can be avoided and/or reduced by closing the slot.

The container is preferably designed as a waste bin. This makes filling easy.

Advantageously, the container and/or the first cavity has a volume in the range of 200 ml or more and/or 200 I or less. These sizes have proven to be particularly practical and suitable.

The container is advantageously set up to be closed in such a way that there are no openings to the outside and/or no passages to the outside, with the exception of an exhaust gas outlet opening from the first cavity out of the container.

The container advantageously has an exhaust gas outlet opening from the first cavity to the outside, in which there is in particular an outlet valve and/or a sterile filter, for example a membrane filter or a depth filter. Through such an exhaust gas outlet opening, in particular after the steam sterilization/decontamination, overpressures can be reduced in a targeted and safe manner and/or exhaust gases can be discharged during the steam sterilization/decontamination. A valve can be used to control this in a targeted manner, and a filter can be used to prevent contamination from escaping, especially at the start of steam sterilization/decontamination.

The exhaust gas outlet opening is particularly advantageously connected to the first cavity with a flexible hose, in particular a TPE hose, and/or the outlet valve is designed as a hose pinch valve. This is a particularly easy and resource-saving way to implement a valve. The exhaust gas outlet opening can also be connected to a valve, for example a diaphragm valve, with the valve being set up to be mechanically controllable and/or mechanically operable, in particular from the outside, in particular by means of the docking station. In particular, the docking station has appropriate means.

The container preferably has no cable bushings and/or no power connections, in particular to the outside, and/or no electrical contacts, in particular to the outside. This increases security.

Apart from an exhaust gas outlet opening, the container advantageously has no connection from the first and/or second cavity to the outside, in particular when the closable slot is closed and/or during steam sterilization/decontamination.

With particular advantage, the docking station has means for generating a negative pressure in the container, in its first cavity, for example a vacuum pump, a blower, a diaphragm pump, a scroll pump, a liquid ring pump, a claw pump and/or it is designed to withstand at least a negative pressure of 0.5 barg. As a result, liquids can be sucked into the first cavity and/or the escape of particles can be reduced.

The container particularly advantageously has means for connecting utilities for generating a negative pressure in the first cavity, e.g. an implementation and/or a tubing for connecting these utilities. The means for connection particularly advantageously have a sterile filter, in particular a hydrophobic one, for example a membrane filter or a depth filter, the means for connection being designed in such a way that air sucked out of the container by the means for connection passes through the sterile filter is conducted.

The at least one second cavity is particularly advantageously divided into a plurality of cavities and/or a plurality of second cavities are provided. In particular, at least one, in particular precisely one, passage and/or at least one second inductively heatable heating means is provided per second cavity and/or per subdivision. The passages are in particular between the first cavity and each second cavity and/or each partition intended. It can thereby be ensured that all passages open, in particular if they open due to excess pressure in the second cavity. The subdivision and/or the at least one second inductively heatable heating means are in particular designed such that sufficient pressure to open the respective passage can be built up in each subdivision by heating by means of the inductively heatable second heating element.

The acquisition in the docking station can be done, for example, by latching and/or at least partially enclosing. The docking station can, for example, have at least one induction coil for generating the electrical, in particular electromagnetic, field for heating an inductively heatable heating element. The means for, in particular supporting, enclosing a container, in particular against bursting in the event of excess pressure in the container up to at least 3 barg and/or as a safety barrier can be formed, for example, by elements for at least partially enclosing the container in a form-fitting manner, for example made of metal. However, they can also be designed to mechanically support the container only in a large number of small areas.

However, the means for enclosing can also be designed solely as protection in the event of the container bursting and/or as thermal insulation and/or have such elements. It is particularly preferred if the container can be completely enclosed by the docking station and/or this is designed for this purpose.

The means for enclosing and/or the docking station can advantageously be designed to be collapsible, sliding and/or foldable, so that the dimensions of the docking station can be reduced by folding, sliding and/or collapsing, in particular so that the dimensions of the docking station in the folded arrangement are finally smaller than the dimensions of the maximal container that can be accommodated by the docking station when ready to use.

The docking station advantageously has at least one sensor, in particular a pressure and/or temperature sensor, which is arranged in such a way that when a container is accommodated and enclosed, in particular with the means for enclosing, the at least one sensor is pressed against the container. A JUMO dTRANS p 31 can be used as a pressure sensor, for example, whose stainless steel membrane is placed and/or pressed from the outside against a flexible plastic membrane of the container.

A PendoTECH “single-use pressure sensor” can be used as a sensor made of “single-use” material, being an integral part of the container.

The docking station preferably has means for receiving wirelessly transmitted measurement data, in particular of the container, and/or has means for wireless power supply of sensors and/or communication means, in particular in the container.

The docking station advantageously has means for sealing and/or marking a container that has been received and/or sterilized/decontaminated. This makes the process particularly safe.

The docking station preferably has means for recognizing and/or reading out an identification of a container and/or storing such an identification. Through this the process can be made safer, especially through documentation of the data.

The docking station particularly advantageously has at least one connection for connecting and/or a receptacle for receiving an exhaust gas outlet of a container that is accommodated. The connection is connected in particular to a sterile filter, for example a membrane filter or a deep-bed filter, in such a way that the air/condensate/steam mixture introduced into the connection is routed through the sterile filter. This makes steam sterilization/decontamination particularly safe. Filters/membranes used, retain contaminants in exhaust gases so that those can be sterilized during the cycle together with the items in the container. In particular, the membrane filters with hydrophobic membrane are guiding the condensate out of the membrane, back to the container.

The docking station, in particular the receptacle, preferably has means for opening and/or closing an exhaust gas valve of the container, in particular the exhaust gas outlet of the container, in particular means for squeezing and/or opening and/or closing a valve of an accommodated container, in particular a flexible one, in particular TPE, tubing and/or tubing pinch valve of the container. In another embodiment, the receptacle and/or the docking station can have a valve, with the connection being connected to the valve in such a way that the air/steam and/or condensate mixture introduced into the connection is routed to the valve. As a result, the steam sterilization/decontamination can be configured particularly safely and carried out particularly reliably.

The docking station is advantageously set up to be surface-decontaminated. For this purpose, it has at least an ingress protection (IP) level of 68. In particular, it has no gaps, no pores and/or no cracks in which contamination can accumulate. It preferably has no open electrical contacts and/or no corroding parts.

With particular advantage, the steam sterilization and/or decontamination and/or heating is carried out exclusively by the inductively heatable heating elements of the container. In particular, the docking station is set up to carry out the steam sterilization/decontamination of the first cavity of the received container exclusively by means of inductively heatable heating elements of the container. In particular, the docking station has no heating means, heaters and/or steam generators. In particular, the container has no heating means and/or steam generators other than the first and/or second inductively heatable heating means.

A possible embodiment of the invention is to be explained below in a purely schematic and non-limiting manner with reference to the following FIGURE.

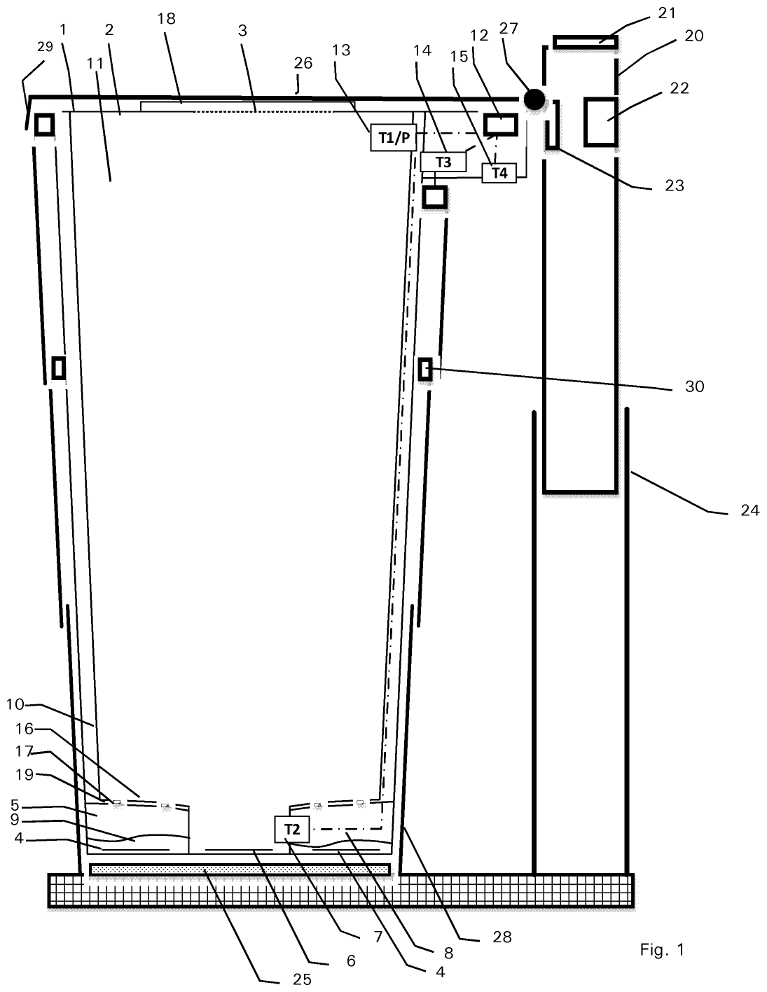

FIG. 1 shows a section through a docking station 20 in which a container 1 is accommodated. A container 1 can be seen, which has a double-wall. The inner wall 10 delimits a first cavity 11 from four sides.

At the bottom, it is delimited by an intermediate floor 19. In the middle of the intermediate floor 19 it is identical to the bottom of the container.

The container has a plurality of second cavities 5 arranged radially. These second cavities are completely closed. In each of them a first heating element 4 is arranged, which can be heated inductively. In addition, water 9 is arranged in every second cavity 5.

In the intermediate floor 19 are arranged for every second cavity 5 valves 17 with nozzles in the direction of the first cavity 11. In addition, the first cavity 11 has an inductively heatable second heating element 6 at its deepest point.

The container 1 has an opening 3 which is closed by means of a closure 18.

In addition, the container 1 has a number of sensors 13, 14, 15 and a wireless communication device 12. The wireless communication device is connected to the sensors via electrically conductive cables. The sensors include a temperature and a pressure sensor 13 for measuring the temperature and the pressure in the upper area of the first cavity 11, a temperature sensor 15 for measuring the temperature in the inner area of the wall of the container 1, a temperature sensor 14 for measuring the temperature of the exhaust air section/on the sterile filter and a temperature sensor 7 for measuring the temperature in the lower area of the first cavity 11. The docking station 20 has a user interface 21 for interaction with the user and a communication device 22 for communication with other control elements, such as tablets and/or network. The communication device 22 is designed in particular for wireless communication.

The docking station 20 is designed to be adjustable in height and for this purpose has a height adjustment 24 with which the height of the docking station 20 can be changed, in particular for reducing the dimensions of the docking station for transport and storage, but it can also be used to adapt to containers 1 of different heights. In addition, the docking station has at least one induction coil 25 for the inductive heating of the heating elements 4, 6.

In addition, the docking station has a wireless communication device 22 for communication with the wireless communication device 12 of the container 1. It also has a cover 26 designed as an insulating wall and support, which can be pivoted via a hinge 27 and at its end opposite the hinge 27 is a locking with the height-adjustable isolation wall 28. The isolation wall 28 also has support extensions 30 to mechanically support a received container 1 and thereby make it more resistant to pressure.

The docking station 20 is set up to heat the inductively heatable heating elements 4, 6 of the container 1 by means of the induction coil 25 and thereby to cause the water 9 to evaporate and to open the valves 17 and to transfer steam into the first cavity 11. The pressure and temperature are measured by the sensors 13, 14, 15 and the measured values are transmitted to the wireless communication device 23 via the wireless communication device 12. In addition, the docking station 20 is set up to transmit energy to the wireless communication device 12 of the container 1 by means of the wireless communication device 23 and thus to transmit the energy for the measurements and the transmission of the measured values. Condensate that collects, for example, on the wall of the first cavity 11 can be routed via the inclined outlet 16 to the second induction heating plate 6 and evaporated there again.

### REFERENCE LIST

1 container
2 lid
3 opening
4 first inductively heatable heating element
5 second cavity
6 second inductively heatable heating element
7 temperature sensor
8 electric line
9 water
10 inner wall
11 first cavity
12 wireless communication device
13 Temperature and pressure sensor
14 temperature sensor
15 temperature sensor
16 outlet
17 valve
18 closure
19 intermediate floor
20 docking station
21 user interface
22 communication facility
23 wireless communication device
24 height adjustment
induction coils
26 cover
27 hinge
28 isolation wall
29 latch
30 support extension

1. A docking station for receiving a container having a first cavity enclosed on all sides, said container having a first inductively heatable heating element, wherein the docking station is set up to generate an electromagnetic field for heating the first inductively heatable heating element of the container; and wherein the docking station further includes means for at least partially enclosing the container against bursting in the event of excess pressure in the container of up to at least 3 barg and/or as a safety barrier.

2. The docking station according to claim 1, wherein the means for at least partially enclosing the container comprises means for thermal insulation of the container.

3. The docking station according to claim 1, wherein the docking station has at least one sensor arranged to press against the container when the container is received in the docking station and is enclosed by the means for at least partially enclosing the containers; or wherein the docking station has means for receiving wirelessly transmitted measurement data or is set up for wirelessly supplying power to sensors in the container.

4. The docking station according to claim 1, wherein the docking station further comprises a connection for connecting or a holder for holding an exhaust gas outlet of the container, said connection being fluidically connected to a sterile filter such that an air/steam/condensate mixture admitted to the connection is directed through the sterile filter; or the holder is designed to open and/or close an exhaust gas valve of the container or has a valve, said connection being fluidically connected to the valve in such a way that an air/steam/condensate mixture admitted to the connection is directed to or through the valve.

5. A system comprising at least one docking station according to claim 1 and at least one container having a first cavity enclosed on all sides, said first cavity including the first inductively heatable heating element.

6. A method for decontamination and/or sterilisation, wherein a container defining a first cavity enclosed on all sides, and having a first inductively heatable heating element, is used for waste collection, and is subsequently brought into a docking station according to claim 1 for sterilisation and/or decontamination, and wherein the container is disposed of after sterilisation and/or decontamination.

7. (canceled)

8. The method according to claim 6, wherein sterilisation and/or decontamination is performed by means of heating the first inductively heatable heating element by the electromagnetic field generated by the docking station.

9. The method according to claim 6, wherein sterilisation and/or decontamination constitutes sterilisation and/or decontamination of the first cavity and/or of material received in the first cavity.

10. (canceled)

11. The method according to claim 6, wherein the docking station is used for sterilisation and/or decontamination of a plurality of containers and/or of the material received therein, wherein each container of the plurality of containers has a first cavity enclosed on all sides, and a first inductively heatable heating element, and wherein the plurality of containers is disposed of after sterilisation and/or decontamination and after each container of the plurality of containers has been removed and/or separated from the docking station.

12. The method according to claim 6, wherein during sterilisation and/or decontamination at least one pressure and/or temperature measured value is received wirelessly from at least one sensor of the container and wherein the electromagnetic field is controlled by the docking station depending on the at least one pressure and/or temperature measured value received.

13. (canceled)

14. A container comprising:
a first cavity enclosed on all sides, said container having a first heating element which is an inductively heatable heating element;
a second cavity adjoining the first cavity or located inside the first cavity; and
wherein the at least one of the first cavity and the second cavity in their entirety is closable and wherein means are provided for transferring steam from the second cavity into the first cavity.

15. The container according to claim 14, further comprising at least one sensor, wherein the at least one sensor is set up for wireless transmission of measured values and/or for wireless power supply, wherein the at least one sensor or its means for transmitting measured values and/or for wireless power supply is/are arranged in an upper half of the container.

16. The container according to claim 14, wherein the container has no power source and/or no means for generating an electromagnetic field and/or no electrical contacts to an environment outside the container.

17. The container according to claim 14, wherein said first cavity and the second cavity are separated from each other by an intermediate floor.

18. The container according to claim 14, wherein the second cavity is at least partially filled with water and/or the container comprises a watertight vessel, arranged on a side of a partition facing away from the first cavity, and/or the watertight vessel is designed such that the watertight vessel opens as a result of temperature and/or the action of heat and/or an increase in pressure caused thereby.

19. The container according to claim 14, further comprising a second inductively heatable heating element or wherein the container is configured such that condensate flowing down an inside of the walls of the container is collected and/or guided to at least one of the first inductively heatable heating element and the second inductively heatable heating element such that the condensate is heated by the at least one of the first inductively heatable heating element and the second inductively heatable heating element.

20. (canceled)

21. The container according to claim 20, further comprising an exhaust gas outlet opening from the first cavity to the environment outside the container, wherein an outlet valve and/or a sterile filter is located in the exhaust gas outlet opening, and/or wherein the exhaust gas outlet opening is connected to the first cavity by a flexible hose or the outlet valve is designed as a hose pinch valve.

22. The container according to claim 14, wherein the second cavity is divided into several cavities and a passage is provided between each second cavity and the first cavity.

23. (canceled)

24. A method for waste disposal, sterilisation and/or decontamination wherein an inductively heatable heating element is heated in a cavity at least partially filled with water by means of a field generated by at least one induction coil to evaporate the water, so that an overpressure is generated in the enclosed cavity and, by opening at least one passage between the cavity and a receiving space by means of the overpressure or by a temperature/heat generated with the inductively heatable heating element, steam is transferred to and/or generated in the receiving space for steam sterilisation of the receiving space and/or contents located in the receiving space.

25. The method according to claim 24, wherein the enclosed cavity and/or the receiving space is/are subsequently incinerated, and wherein the at least one induction coil is used to heat another inductively heatable heating element.
